(19) United States
(12) Patent Application Publication
(10) Pub. No.: US 2004/0082532 A1
(43) Pub. Date: Apr. 29, 2004

(54) Antifungal therapeutic targets

(76) Inventors: Christopher J. Savoie, Fukuoka (JP); Satoru Kuhara, Fukuoka (JP); Kosuke Tashiro, Fukuoka (JP)

Correspondence Address: GRAY CARY WARE & FREIDENRICH LLP, 153 TOWNSEND, SUITE 800, SAN FRANCISCO, CA, 94107, US

(21) Appl. No.: 10/620,149

(22) Filed: Jul. 14, 2003

Related U.S. Application Data
(60) Provisional application No. 60/395,756, filed on Jul. 12, 2002.

(51) Int. Cl. A61K 48/00; A61K 31/496

(52) U.S. Cl. 514/44; 514/254.07

(57) ABSTRACT

The present invention provides therapeutic targets for antifungal treatment, especially CIK1.

### RELATED APPLICATION

The present application claims priority under 35 U.S.C. §119(e) from provisional application No. 60/395,756, filed Jul. 12, 2002.

### FIELD OF THE INVENTION

This invention relates generally to the field of fungal infection, and more specifically to targets suitable for antifungal treatments.

### BACKGROUND OF THE INVENTION

Fungal infections are common in mammals, especially in humans. There is a need in the art to provide compositions or methods useful for treating or preventing fungal infections, e.g., dermatophytic infections.

### SUMMARY OF THE INVENTION

The present invention is based on the discovery that therapeutic targets can be rationally identified using network model analysis. Accordingly the present invention provides various groups of therapeutic targets, pathways, and the uses thereof for antifungal treatments.

In one embodiment, the present invention provides a method of affecting an antifungal activity in a system. The method comprises administering to the system an agent, wherein the agent affects a target gene whereby affecting the antifungal activity in, the system, and wherein the target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W.

In another embodiment, the present invention provides a method of increasing the antifungal activity in a system. The method comprises administering to the system an agent, wherein the agent decreases the activity of a target gene selected from the group consisting of CIK1, YFLO54C, SKN7, DDR48, LEU3, FUS1, and GZF3.

In yet another embodiment, the present invention provides a method of increasing the antifungal activity in a systems. The method comprises administering to the system an agent, wherein the agent affects a target gene involved in the CIK1 pathway whereby decreasing the activity of CIK1, wherein the target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1,PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W.

In yet another embodiment, the present invention provides a database. The database comprises a plurality of target genes corresponding to an antifungal agent, wherein each target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJl, YCK2, KAR3, SPT4, CRZ1, ZAP1, DAL5, PEX11, HTA1, ZIP1, HTA2, BAR1, GAL80, and YDL182W.

In yet another embodiment, the present invention provides an isolated polynucleotide. The isolated polynucleotide comprises a target sequence consisting of a partial sequence of a target gene, wherein the target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W, and wherein the activity of the partial sequence of the target gene is responsive in a cell to an antifungal agent.

In yet another embodiment, the present invention provides a system containing a plurality of samples, wherein each sample is a target gene selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W, and wherein the system allows for parallel analysis of each target gene.

In yet another embodiment, the present invention provides a method of screening for a candidate antifungal agent. The method comprises contacting a target with a test agent, wherein the target is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W, determining the activity of the target, wherein a change of the activity caused by a test agent is indicative of the test agent as a candidate antifungal agent.

### DESCRIPTION OF THE PREFERRED EMBODIMENTS

The present invention relates in general to therapeutic targets useful for antifungal treatment. It is the discovery of the present invention that various groups of genes and pathways can be used as targets for antifungal treatment. Accordingly, the present invention provides target genes and their pathways, individually or as a database or system, useful for identifying antifungal agents. The present invention also provides methods of affecting antifungal activity in a system by affecting target genes provided by the present invention.

One feature of the present invention provides polynucleotides encoding target genes for antifungal treatment. The polynucleotide provided by the present invention includes a target sequence containing a partial sequence of a target gene, and optionally a sequence heterologous to the target gene. The target sequence can include one or more partial sequences from one or more target genes provided by the present invention. The partial sequence of a target gene can include a portion or full-length of the target gene. In one embodiment, the target sequence includes full-length of the target gene whose specific, credible and substantial utility is not known prior to Jul. 12, 2002.

In another embodiment, the target sequence includes a portion of a target gene encoding an activity responsive to an antifungal agent in a cell, e.g., in yeast. For example, the-portion of a target gene included in the target sequence can encode or provide an activity that changes upon encountering, directly or indirectly, to an antifungal agent. The activity encoded or provided by such portion can be any activity, known or to be discovered, that is associated with a cell's or system's response to an antifungal agent, or a fungal infection.

The partial sequence of a target gene included in the target sequence can be from any target gene of an antifungal agent. In one embodiment, the target gene is a direct target of an antifungal agent, e.g., griseofulvin and can be AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, or LEU3.

In another embodiment, the target gene encodes a receptor such as AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

In yet another embodiment, the target gene encodes a gene directly associated with a receptor, e.g. KAR3, SPT4, CRZ1, ZAP1, DAL5, PEX11, HTA1, ZIP1, HTA2, ZAP1, BAR1, GAL80, and YDL182W. In still another embodiment, the target gene is CIK1, YFLO54C, SKN7, DDR48, LEU3, FUS1, or GZF3.

In still yet another embodiment, the target gene is CIK1 and receptors associated with CIK1, e.g., MFALPHA2, GAL11, HSP82, KAR2, SNI2, FAR1, SNF6, AFR1, GPA1, STE2, and STE4, or genes directly associated with such, receptors, e.g., KAR3, SPT4, CRZ1, ZAP1, DAL5, PEX11, HTA2, ZIP1, HTA2, BAR1, GAL80, and YDL182W.

According to the present invention, the target gene provided by the present invention includes the target gene encoded in any organism, e.g., microorganisms. For example, the target gene can be encoded in various species of fungi including, without limitation, Trichophyton, e.g., T. rubrum, T. mentagrophytum and T. interdigitale, Microsporum Canis, and Candida, e.g., Candida albicans, C. glabrata, C. guilliemondii, C. kefyr, C. krusei, C. stellatoidea and C. tropicalis. The target gene can also include homologues of the target gene and target genes containing one or more mutations or polymorphisms, e.g., SNPs.

According to another feature of the present invention, it provides polypieptides or crystalline polypeptides encoded by the polynucleotides of the present, invention. The present invention also provides cells, e.g., eukaryotic or prokaryotic cells and vectors, e.g., expression vectors containing polynucleotides or polypeptides encoded by the polynucleotides provided by the present invention. In addition, the present invention provides antibodies that are capable of specifically binding to the polypeptides of the present invention and modulating one or more activities of the polypeptides, e.g., activities associated with a fungal infection, gene pathway.

According to another feature of the present invention, it provides databases containing the target genes provided by the present invention. In one embodiment, the database provided by the present invention contains two or more target genes provided by the present invention.

In another embodiment, the database provided by the present invention contains two or more target genes and each target gene is assigned an identity identifier and a relationship identifier. The identity identifier identifies each target gene, whereas the, relationship identifier identifies each target gene's relationship to another target gene. For example, a relationship identifier can identify how each target gene relates to other target genes in the database, e.g., the gene pathways and the level of such relationship, e.g., directly, secondary, etc.

In yet another embodiment, the database provided by the present invention is in a computer readable medium, e.g., can be accessed on site or remotely. In still another embodiment, the present invention provides a user interface operatively working with a processor to affect operation of the database provided by the present invention. The user interface can include a display area displaying the relationship of two or more target genes within the database.

The target genes provided by the present invention can also be included in a system useful for parallel analysis of each target gene. In one embodiment, the present invention provides a system containing two or more target genes provided in the present invention. In another embodiment, the present invention provides a system containing two or more polynucleotides or polypeptides including crystalline polyp ides provided by the present invention. In yet another embodiment, the present invention provides a system containing two or more samples containing cells or vectors having the polynucleotides or polypeptides provided by the present invention. The system provided by the present invention is useful for performing parallel processing or analysis of each target gene, e.g., a high throughput system.

According to another feature of the present invention, the target genes including the polynucleotides, polypeptides, and crystalline polypeptides provided by the present invention can be used for drug discovery and design. For example, the crystalline polypeptides provided by the present invention can be used as a guide for identifying agents that are capable of affecting the activity of the polypeptides, e.g., identify inhibitors or enhancers of the polypeptides of the present invention. In one embodiment, the structure coordinates or atomic coordinates of the polypeptide of the present invention are used to design a potential inhibitor or enhancer that will form a covalent or non-covalent bond with one or more amino acids of the polypeptide of the present invention.

The structure or atomic coordinates of the polypeptide of the present invention refer to mathematical coordinates derived from mathematical equation's related to the patterns obtained on diffraction of a monochromatic beam of X-rays by the atoms (scattering centers) of the polypeptide in crystal form. The diffraction data normally, are used to calculate an electron density map of the repeating unit of the crystal. The electron density maps are generally used to establish the positions of the individual atoms within the unit cell of the crystal.

Various methods can be used to obtain the structure or atomic coordinates of the polypeptides provided by the present invention. For example, three dimensional diffraction data for a polypeptide of the present invention can be collected at temperatures ranging from 100-274 K using an area detector and radiation from a rotating-anode X-ray generator and from the Stanford synchrotron. These data, along with data collected from a heavy atom derivative of the polypeptide, can be processed and the structure can be solved by methods which make use of the isomorphous differences between a derivative and native polypeptide and/or make use of the anomalous X-ray scattering from the heavy atom in the derivative.

According to another feature of the present invention, the target genes including the polynucleotides and polypeptides provided by the present invention can be used in screening assays as a target to identify inhibitors or enhancers of the target genes provided by the present invention, e.g., candidates for antifungal agents. For example, a test agent can be contacted, either directly or indirectly, with a target, e.g., in vitro or in vivo. Any change of an activity of the target caused by the test agent is indicative of the test agent as an antifungal agent.

The target used in the screening assays can be any form of the target genes provided by the present invention. In one embodiment, the target used in the screening assays is a target gene such as AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W or one or more portions thereof.

In another embodiment, the target used in the screening assay is AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, or YDL182W, or one or more portions thereof.

In yet another embodiment, the target used in the screening assay is CIK1, YFLO54C, SKN7, DDR48, LEU3, FUS1, or GZF3, or one or more portions thereof. In still another embodiment, the target used in the screening assay is the polynucleotides or polypeptides provided by the present invention.

Normally a change of an activity of the target used in the screening assays includes an increase or decrease of any assayable activity of the target. In one embodiment, the activity of the target is the expression level of the target. In another embodiment, the activity of the target, is the target's ability to specifically interact with a test agent, e.g., binding activity. In yet another embodiment, the activity of the target is an activity associated with an antifungal agent or a fungal infection. test agent used for the screening methods of the present invention can be any agent from any library of compounds or molecules. For example, the test agent can be any polypeptide, polynucleotide, compound, small molecule, or antibody.

According to another feature of the present invention, it provides methods of affecting antifungal activity in a system, e.g., in vitro or in vivo. For example, the antifungal activity or a fungal infection response in a system, e.g., human can be modulated via modulating one or more target genes provided by the present invention.

In one embodiment, the antifungal activity of a system can be affected or modulated by affecting one or more target genes such as AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W.

In another embodiment, the antifungal activity of a system can be affected or modulated by affecting one or more target genes including AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, and LEU3.

In yet another embodiment, the antifungal activity of a system can be affected or modulated by affecting one or more receptor genes, e.g., AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

In still another embodiment, the antifungal activity of a system can be affected or modulated by affecting genes directly associated with receptor genes, e.g., KAR3, SPT4, CRZ1, ZAP1, DAL5, PEX11, HTA1, ZIP1, HTA2, BAR1, GAL80, or YDL182W.

In still yet another embodiment, the antifungal activity of a system can be affected or modulated by affecting CIK1 or one or more genes including CIK1, YFLO54C, SKN7, DDR48, LEU3, FUS1, and GZF3.

According to the present invention, any suitable means can be used to affect, directly or indirectly, the target genes of the present invention. For example, an agent can be administered to a system to block or enhance, completely or partially, one or more functional sites, e.g., binding or activation sites of a target gene. Alternatively, an agent can be administered to a system to inhibit or enhance the activity of a gene which is upstream or downstream of a desired target gene. The agent used in the methods provided by the present invention can be any agent, e.g., suitable therapeutic agent including a known agent or an agent to be discovered. In one embodiment, the agent used in the methods provided by the present invention does not include any anti-fungal agent known prior to Jul. 12,.2002.

In another embodiment, the present invention provides a method of increasing the antifungal activity in a system in vitro or in vivo by decreasing the activity of one or more target genes. For example, the antifungal activity of a system can be increased by decreasing the activity of a target gene such as CIK1, YFLO54C, SKN7, DDR48, LEU3, FUS1, and GZF3. Alternatively the antifungal activity of a system can be increased by affecting genes involved in the target gene pathways of CIK1, YFLO54C, SKN7, DDR48, FUS1, and GZF3, whereby decreasing the activity of CIK1, YFLO54C, SKN7, DDR48, FUS1, and GZF3, respectively.

According to another embodiment of the present invention, the antifungal activity of a system can be increased by decreasing CIK1 activity directly or through its pathway, e.g., via affecting receptor genes in its pathway including AFR1, ARP7, AXL1, CRM1, CSE2, CSE1, FAR1, GPA1, GAL1, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

In yet another embodiment, the antifungal activity of a system can be increased by decreasing CIK1 activity via affecting genes directly associated with the receptor genes in CIK1 pathway, e.g. via affecting KAR3, SPT4, CRZ1, ZAP1, DAL5, PEX11, HTA1, ZIP1, HTA2, BAR1, GAL80, or YDL182W.

The agents of the present invention for modulating or affecting antifungal activity in a system can be administered alone, in a composition with a suitable pharmaceutical carrier, or in combination with other therapeutic agents. An effective amount of the agents to be administered can be determined on a case-by-case basis. Factors to be, considered usually include age, body weight, stage of the condition, other disease conditions, duration of the treatment, and the response to the initial treatment.

Typically, the compositions containing the agents are prepared as a topical or an injectable, either as a liquid solution or suspension. However, solid forms suitable for solution in, or suspension in, liquid vehicles prior to injection can also be prepared. The composition containing the agent can also be formulated into an enteric-coated tablet or. gel capsule according to known methods in the art.

The compositions containing the agents used in the present invention may be administered in any way which is medically acceptable which may depend on the disease condition or injury being treated. Possible administration routes include injections, by parenteral routes such as intravascular, intravenous, intraepidural or others, as well as oral, nasal, ophthalmic, rectal, topical, or pulmonary, e.g., by inhalation. The compositions may also be directly applied to tissue surfaces. Sustained release, pH dependent release, or other specific chemical or environmental condition mediated release administration is also specifically included in the invention, by such means as depot injections or erodible implants.

Although the invention has been described with reference to the presently preferred embodiment, it should be understood that various modifications can be made without departing from the spirit of the invention. Accordingly, the invention is limited only by the following claims.

What is claimed is:

1. A method of affecting an antifungal activity in a system comprising administering to the system an agent, wherein the agent affects a target gene whereby affecting the antifungal activity in the system, and wherein the target gene is selected from the group consisting of AAC3, ABF1, CDC6, CIK1, COQ7, CPA2, DDR48, FAS1, FUS1, GAL80, GDH2, GRF10, GZF3, HSP12, IME1, TPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W.

2. The method of claim 1, wherein the target gene is selected from the group consisting of CIK1, YFLO54C, SKN7, DDR48, LEU3, FUS1, and GZF3.

3. The method of claim 1, wherein the target gene is CIK1.

4. The method of claim 1, wherein the agent inhibits the activity of CIK1 and whereby the antifungal activity, in the system is increased.

5. The method of claim 1, wherein the target gene is selected from the group consisting of AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

6. The method of claim 1, wherein the target gene is selected from the group consisting of KAR3, SPT4, CRZ1, ZAP1, DAL5, PEX11, HTA1, ZIP1, HTA2,

7. A method of increasing the antifungal activity in a system comprising administering to the system an agent, wherein the agent decreases the activity of a target gene selected from the group consisting of CIK1, YFLO54C, SKN7, DDR48, LEU3, FUS1, and GZF3.

8. A method of increasing the antifungal activity in a system comprising administering to the system an agent, wherein the agent affects a target gene involved in the CIK1 pathway whereby decreasing the activity of CIK1, wherein the target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, APR7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2. STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W.

9. The method of claim 8, wherein the target gene is selected from the group consisting of a AFR1, ARP7, AXL1, CRM1, CSE2, CSE1, FAR1, GPA1, GAL11, HSP82, IPL1, KAR2, MFALPHA2, MSN25, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

10. The method of claim 8, wherein the target gene is selected from the group consisting of KAR3, SPT4, CRZ1, ZAP1, DAL5, PEX11, HTA1, ZIP1, HTA2, BAR1, GAL80 and YDL182W.

11. A database comprising a plurality of target genes corresponding to an antifungal agent, wherein each target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W.

12. The database of claim 11, wherein each target gene is assigned a first identifier identifying its identity and a second identifier identifying its relationship with a target gene in the database.

13. An isolated polynucleotide comprising a target sequence consisting of a partial sequence of a target gene, wherein the target gene is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W, and wherein the activity of the partial sequence of the target gene is responsive in a cell to an antifungal agent.

14. The isolated polynucleotide of claim 13, wherein the target sequence is adjacent to a heterologous sequence.

15. The isolated polynucleotide of claim 13, wherein the target gene is selected from the group consisting of CIK1, YFLO54C, SKN7, DDR48, LEU3, FUS1, and GZF3.

16. The isolated polynucleotide of claim 13, wherein the target gene is CIK1.

17. The isolated polynucleotide of claim 13, wherein the target gene is selected from the group consisting of AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

18. The isolated polynucleotide of claim 13, wherein the target gene is selected from the group consisting of KAR3, SPT4, CRZ1, ZAP1, DAL5, PEX11, HTA1, ZIP1, HTA2, BAR1, GAL80, and YDL182W.

19. An isolated polypeptide encoded by the polynucleotide of claim 13.

20. An antibody binding specifically to the polypeptide of claim 19, wherein the antibody modulates an activity of the polypeptide and wherein the activity is associated with a fungal infection.

21. A vector containing the polynucleotide of claim 13.

22. A cell containing the polynucleotide of claim 13.

23. A cell containing the vector of claim 21.

24. An isolated crystalline polypeptide comprising the polypeptide of claim 19.

25. A system containing a plurality of samples, wherein each sample is a target gene elected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W, and wherein the system allows for parallel analysis of each target gene.

26. A system containing a plurality of samples, wherein each sample is a polynucleotide of claim 13 and wherein the system allows for parallel analysis of each sample.

27. A system containing a plurality of samples, wherein each sample is a polypeptide of claim 19 and wherein the system allows for parallel analysis of each sample.

28. The system of claim 25, wherein the system is a high throughput system.

29. The system of claim 26, wherein the system is a high throughput system.

30. The system of claim 27, wherein the system is a high throughput system.

31. A method of screening for a candidate antifungal agent comprising
contacting a target with a test agent, wherein the target is selected from the group consisting of AAC3, ABF1, CDC6, CIK1, COQ7, CPA2, DDR48, FAS1, FUS1, GAL80, GDH2, GRF10, GZF3, HSP12, IME1, IPT1, MAL33, PCL9, PGM2, PHO23, POB1, PPR1, PTP1, SOD2, TRR1, TSA2, UGA1, YFL054C, SKN7, LEU3, AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, YCK2, KAR3, SPT4, CRZ1, ZAP1, DAL5, PEX11, HTA1, ZIP1, HTA2, BAR1, GAL80, and YDL182W,
determining the activity of the target,
wherein a change of the activity caused by a test agent is indicative of the test agent as a candidate antifungal agent.

32. The method of claim 31, wherein the activity of the target is the level of the target in a cell.

33. The method of claim 31, wherein the activity of the target is the ability of the target to bind specifically to a test agent.

34. The method of claim 31, wherein the activity of the target is an activity associated with a cell's response to an antifungal agent.

35. The method of claim 31, wherein the target is selected from the group consisting of CIK1, YFLO54C, SKN7, DDR48, LEU3, FUS1, and GZF3.

36. The method of claim 31, wherein the target is selected from the group consisting of AFR1, ARP7, AXL1, CRM1, CSE2, CSE1, FAR1, GPA1, GAL11, HSP82, IPL1, KAR2, MFALPHA2, MSN5, MTR10, NPL3, PSU1, RPS0B, SEC72, SLA2, SNI2, SNF5, SNF6, SNC2, SRP1, SRB4, STE2, STE4, SUP35, SWI3, UBI3, UBI2, VAM3, YDJ1, and YCK2.

37. The method of claim 31, wherein the target is selected from the group consisting of KAR3, SPT4, CRZ1, ZAP1, DAL5, PEX11, HTA1, ZIP1, HTA2, BAR1, GAL80, and YDL182W.

38. The method of claim 31, wherein the target is CIK1.

39. The method of claim 31, wherein the target is in a cell.

40. An antifungal agent identified by the method of claim 31.
